(19) United States
(12) Plant Patent Application Publication
(10) Pub. No.: US 2018/0199482 P1
(43) Pub. Date: Jul. 12, 2018

(54) Vaccinium corymbosum L. plant named 'RYOKU NH-12'

(50) ;

(71) Applicant: NIPPON RYOKUSAN CO., LTD., Matsumoto-shi (JP)

(72) Inventor: Shigetaka Sakurai, Matsumoto-shi (JP)

(73) Assignee: NIPPON RYOKUSAN CO., LTD., Matsumoto-shi (JP)

(21) Appl. No.: 15/732,725

(22) Filed: Dec. 21, 2017

(30) Foreign Application Priority Data
Jan. 4, 2017 (JP) 31724

(51) Int. Cl. A01H 5/08

(52) U.S. Cl. PLT/157

(57) ABSTRACT

A new and distinct variety of Vaccinium corymbosum L. plant named ‘RYOKU NH-12’, characterized by having more compact plant size, comparatively early fruit ripening time, comparatively large and uniform fruit size, firmer and lower dehiscent fruits, sweeter fruits, better taste balance of sweetness and acidity, and better taste of fruits, as compared to other Vaccinium corymbosum L. varieties.

### CROSS-REFERENCE TO RELATED APPLICATIONS

This application claims priority to Japanese Plant Breeders' Rights Application No. 31724, filed Jan. 4, 2017, the contents of which are incorporated herein by reference in their entirety.

The Latin name of the genus and species of the novel variety disclosed herein is: Vaccinium corymbosum L.

The novel variety of the Vaccinium corymbosum L. disclosed herein has been given the variety denomination: ‘RYOKU NH-12’.

### BACKGROUND OF THE INVENTION

The present invention relates to a new and distinct hybrid variety of northern highbush blueberry (Vaccinium corymbosum L.) named ‘RYOKU NH-12’. This novel variety was found by open pollination of ‘Spartan’, ‘Duke’ and ‘Denies Blue’ in the tests conducted for the period from 2004 to 2008 in Matsumoto-City, Nagano-prefecture, Japan. As stated below, ‘RYOKU NH-12’ has apparently different characteristics from those of the ‘Spartan’ and ‘Duke,’ both being widely planted and important varieties in the Chubu district of Japan.

### SUMMARY OF THE INVENTION

Blueberry variety ‘RYOKU NH-12’ exhibits outstanding and distinguishing characteristics when grown under normal horticultural conditions in the area from Nagano-prefecture to the north of the Kanto in Japan, including:
(1) more compact plant size;
(2) comparatively early fruit ripening time (50% fruit ripening around late June of each year in Matsumoto, Nagano, Japan);
(3) comparatively large and uniform fruit size;
(4) firmer and lower dehiscent fruits;
(5) sweeter fruits with better taste balance of sweetness and acidity; and
(6) better taste of fruits.

### BRIEF DESCRIPTION OF THE DRAWINGS

The accompanying colored photographs (FIGS. 1 to 8) show typical bush, flower, fruit and leaf characteristics of the new Vaccinium corymbosum L. plant, ‘RYOKU NH-12’. Colors shown are as accurate as can be reasonably reproduced by photographic means. In some cases, the color might differ slightly from the colors of ‘RYOKU NH-12’ recited in the description.

FIG. 1 shows a tree body of ‘RYOKU NH-12’ (photographed date: Jul. 9, 2014; photographed location: NIPPON RYOKUSAN CO., LTD, Matsumoto-City, Nagano-prefecture, Japan).

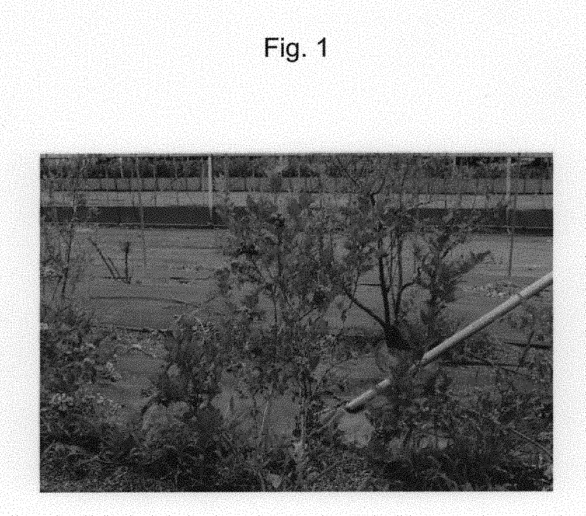

FIG. 2 shows a panoramic view of the cultivation area of ‘RYOKU NH-12’ (photographed date: Sep. 7, 2012; photographed location: same as FIG. 1).

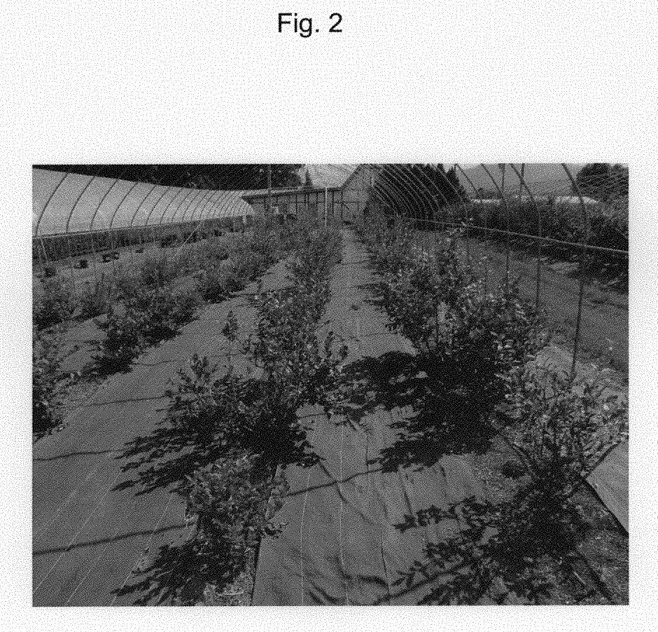

FIG. 3 shows whole flowers of ‘RYOKU NH-12’ (photographed date: May 6, 2014; photographed location: same as FIG. 1).

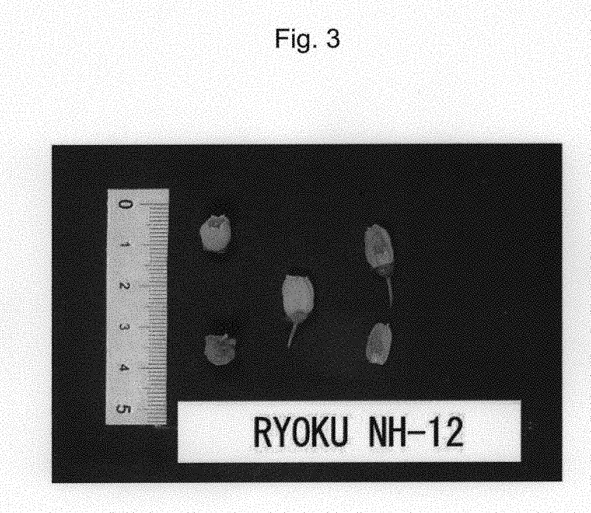

FIG. 4 shows broken-down parts of a flower of ‘RYOKU NH-12’ (photographed date: May 6, 2014; photographed location: same as FIG. 1).

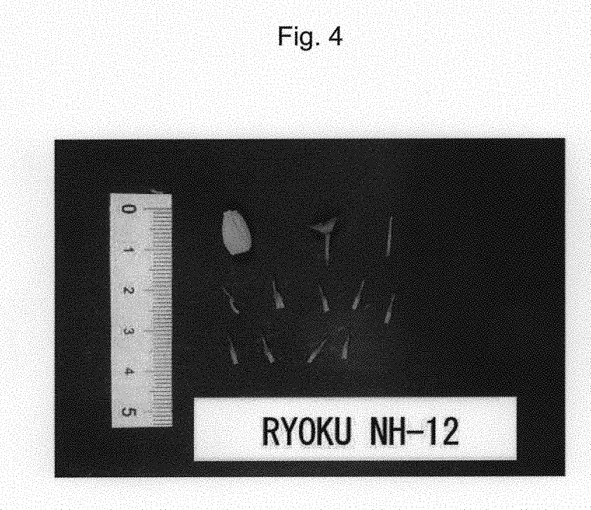

FIG. 5 shows fruits of ‘RYOKU NH-12’ (photographed date: Jul. 15, 2014; photographed location: same as FIG. 1).

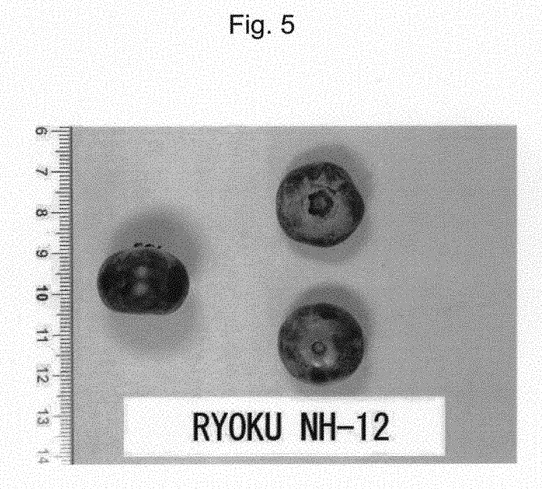

FIG. 6 shows a cross-section of the fruits of ‘RYOKU NH-12’ (photographed date: Jul. 15, 2014; photographed location: same as FIG. 1).

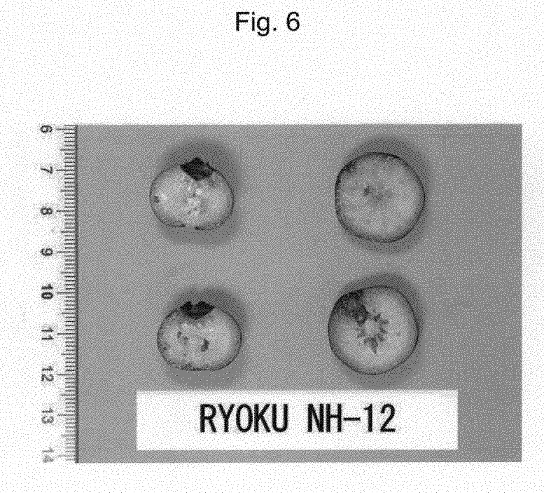

FIG. 7 shows an upper side of the leaves (10 sheets) of ‘RYOKU NH-12’ (photographed date: Aug. 3, 2012; photographed location: same as FIG. 1).

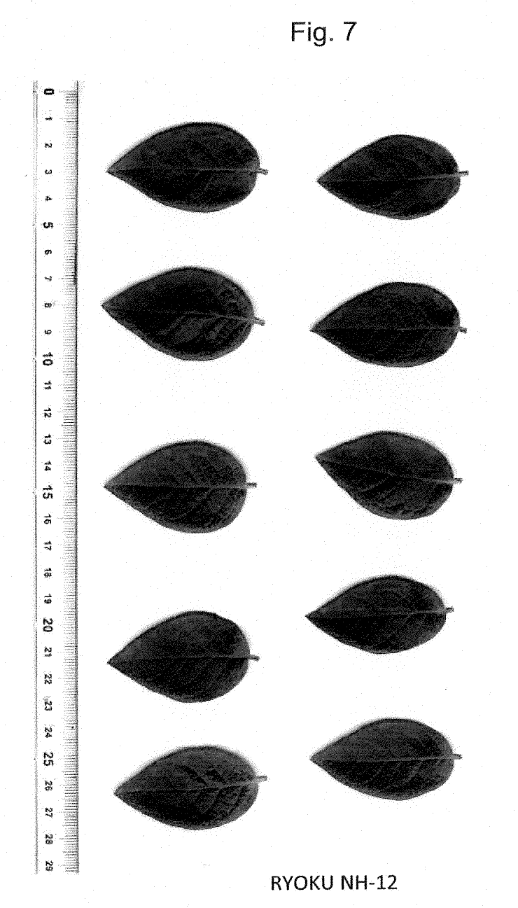

FIG. 8 shows a lower side of the leaves (10 sheets) of ‘RYOKU NH-12’ (photographed date: Aug. 3, 2012; photographed location: same as FIG. 1).

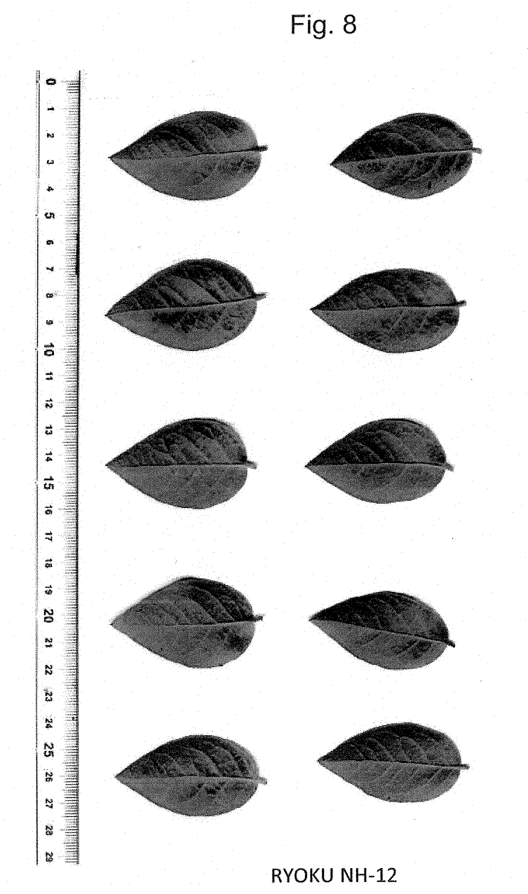

### DETAILED BOTANICAL DESCRIPTION

### A. Distinctive Characteristics of ‘RYOKU NY-12’

As described above, ‘RYOKU NH-12’ was obtained by the open pollination of ‘Spartan’, ‘Duke’ and ‘Denies Blue.’ On Apr. 15, 2004, about 1,000 seeds obtained from the mixed fruits of ‘Spartan’, ‘Duke’ and ‘Denies Blue,’ all of which were harvested in the field of NIPPON RYOKUSAN CO., LTD. (Matsumoto, Nagano, Japan), seeded in plant seeding trays, and then transplanted to pots. The resulting seedlings (about 300) were planted in cultivation pots on May 1, 2005. Fructification of the planted seedlings was started from Jun. 20, 2008 (on Year 3), and about 20 plant individuals were selected based on the characteristics, including larger fruit size, better taste balance of sweetness and acidity, higher yield per plant, earlier ripening time, large and uniform fruits, etc. For the about 20 plant individuals selected, test plots (each including about 20 cuttings per plant individual) were formed, where these plants were asexually propagated by cutting means. During the period from Jul. 20, 2010 to Dec. 25, 2016 and for 3 generations, the plants were propagated and examined for their characteristics based on the growth, and yield and fruits quality in each test plot. For 10 test plots, the characteristics of the plants were observed for the period between the flowering time and the ripening time yearly for 4 years, and since neither variant nor off-type plant was observed for the period, the characterization of ‘RYOKU NH-12’ was finished on Dec. 25, 2016 and the breeding was completed.

‘RYOKU NH-12’ is a blueberry clone distinguishable from the important blueberry varieties ‘Spartan’ and ‘Duke’, both of which are widely planted in the Chubu district of Japan, due to its characteristics including more compact plant size, comparatively early fruit ripening time, comparatively large and uniform fruit size, firmer and lower dehiscent fruits, sweeter fruits with better taste balance of sweetness and acidity, and better taste fruits. 20 plants of ‘RYOKU NH-12’ had been propagated by cutting means in Matsumoto, Nagano, Japan, and all the resulting plants were phenotypically indistinguishable from the original plant variety ‘RYOKU NH-12’. In addition, comparing to ‘Spartan’ and ‘Duke’, the claimed plant ‘RYOKU NH-12’ has more compact plant size, about 1 week earlier fruit ripening time (on average, around June 25 to July 1 of each year in Matsumoto, Nagano, Japan), larger and more uniform fruit size, and firmer and sweeter fruit when compared to its related variety ‘Spartan’, and has more compact plant size, lower dehiscent fruits, sweeter fruits, a better balance of sweetness and acidity, and better tasting of fruits when compared to the ‘Duke’ variety (see Table 1 below).

The following data defining characteristics of ‘RYOKU NH-12’ were collected from the asexual propagation carried out in Matsumoto, Nagano, Japan. The plant history was taken on a plot of 10 four-year-old plants growing in Matsumoto, Nagano, Japan. ‘RYOKU NH-12’ has not been observed under all possible environmental conditions, and the measurements provided might therefore vary if grown in different environments. Where averages are given, the sample size was 10.

B. Phenotypic Description of Vaccinium corymbosum L. (‘RYOKU NH-12’)

Characteristics of ‘RYOKU NH-12’ are further specifically described as follows:
1. Plant:
   Plant vigor.—Medium.
   Growth habit.—Semi-upright.
   Plant size.—Small.
   Plant height.—1.3 m on average for 4-year old plant.
   Plant spread.—0.8 m on average for 4-year old plant.
   Cold hardiness.—Survived in winter frost (below —10° C.) with minimum damage.
   Ease of propagation.—Propagated from each of the dormant wood cutting and the softwood stem cutting, where the rooting percentage was greater than 70% and comparable to the other varieties.
2. Trunk and branches:
   Suckering tendency.—Medium suckering tendency.
   Surface texture (of 6-month-old shoots).—Medium smoothness.
   Surface texture (of 3-year-old and older wood).—Medium smoothness.
   Color of new twigs observed in the field.—Orangish yellow green.
   Internode length.—16.2 mm on average.
3. Leaves:
   Length including petiole.—54.0 mm on average.
   Width of leaf at widest point.—33.0 mm on average.
   Shape.—Ovate.
   Leaf margin.—Entire.
   Color.—Upper surface: Medium Green.
   Pubescence.—Upper Surface of leaves: Absent. Lower Surface of leaves: Absent. Margin: Absent.
   Timing of vegetative bud burst.—Medium.
4. Flowers:
   Shape.—Campanulate.
   Flowering period.—Mean date of 50% opening of flowers in Matsumoto-City, Nagano-prefecture, Japan is May 1 (2 days earlier than ‘Spartan’ variety).
   Corolla.—Diameter: 7.4 mm on average. Length (from pedicel attachment point to corolla tip excluding the pedicel): 10.8 mm on average. Color: light green white. Anthocyanin coloration in corolla tube — Absent or very weak.
5. Reproductive organs:
   Pollen.—Color: Yellow.
6. Fruit:
   Mean date of 50% harvest in Matsumoto-City, Nagano-prefecture.—July 1 on average.
   Diameter of calyx aperture on mature berry.—5.9 mm on average.
   Size and shape of calyx lobe on mature berry.—Medium in size, outcurving, and having deep calyx basin.
   Detachment force for ripe berries (easy, medium, hard).—Easy.
   Fruit cluster density (sparse, medium, dense).—Medium.
   Fruiting type.—On one-year old shoots only.
7. Berry:
   Cluster (tight, medium, loose).—Medium.
   Weight (on well-pruned plants).—3.79 g on average.
   Height.—14.9 mm on average.
   Width.—20.4 mm on average.
   Shape.—Oblate.
   Surface color of mature berries ripe on the plant.—Blue purple.
   Intensity of fruit bloom.—Medium.
   Surface color of ripe berry after polishing.—Medium blue.
   Immature berry color, with bloom.—Medium green.
   Immature berry color, without bloom.—Yellow green.
   Flesh color.—White.
   Peel color.—Medium blue.
   Pedicel scar.—Medium. 2.92 mm on average.
   Firmness.—Medium.
   Intensity of fruit sweetness.—High, Bx 14.5.
   Intensity of fruit acidity.—High, pH 2.59.
   Texture.—Somewhat soft pulp, very juicy, medium seeds.
8. Use: ‘RYOKU NH-12’ produce northern highbush blueberries suitable for fruit-picking farms, fresh fruit markets and processed fruit market, etc.
9. Resistance to disease, insects, and mites: ‘RYOKU NH-12’ grew vigorously and showed excellent bush survival in the field. It appears to be tolerant to stem blight (Botryosphaeria spp.) and root rot (Phytophthora cinnamoni), with very few young plants dying soon after planting. The response of ‘RYOKU NH-12’ to the various fungal species that cause summer leaf spots is typical of other northern highbush varieties, and fungicide applications may be needed after harvest in order to reduce foliar diseases and to retain leaves until autumn and make maximum flower bud set. Similarly, susceptibility to typical blueberry insect and mite pathogens, such as spotted wing drosophila (Drosophila suzukii), blueberry gall midge (Dasineura oxycoccana) and blueberry bud mite (Acalitus vaccini), is similar to other northern highbush cultivars.

TABLE 1
(Comparison of characteristics among varieties)

| | | | | | | | | | The | | |
|---|---|---|---|---|---|---|---|---|---|---|---|
| | | | | | | | | | present | Control | |
| Char- | | | | | | | | Standard | variety | | |
| act. | UPOV | | | | | | | Variety | RYOKU | Varieties | |
| No. | No. | Code | Characteristics | Definition | Method | Class | State | (Ex. Var.) | NH-12 | Spartan | Duke |
| 1 | 1 | QN | Plant: vigor | Stength of growth | Observa- | 3 | weak | Bluetta, | | | |
| | (*) | (+) | | level of plant | tion | | | Meader | | | |
| | | | | | (a) | 5 | medium | Collins, | | | |
| | | | | | VG | | | Weymouth | 5 | 6 | 7 |
| | | | | | | 7 | strong | Berkeley, | | | |
| | | | | | | | | Homebell, | | | |
| | | | | | | | | Woodward | | | |
| 2 | | QN | Plant: size | Size of plant crown | Observa- | 3 | small | Avonblue, | | | |
| | | | | | tion | | | Bluetta, | | | |
| | | | | | (a) | | | Flordablue | | | |
| | | | | | VG | 5 | medium | Bluecrop, | 3 | 5 | 7 |
| | | | | | | | | Earliblue | | | |
| | | | | | | 7 | large | Dixi, | | | |
| | | | | | | | | Homebell, | | | |
| | | | | | | | | Tifblue | | | |
| 3 | 2 | QN | Plant: growth habit | Whole shape of | Observa- | 1 | upright | Becyblue, | | | |
| | (*) | G | | plant without | tion | | | Bluechip, | | | |
| | | | | pruning during | (a) | | | June, | | | |
| | | | | dormant period | VG | | | Spartan | 2 | 1 | 1 |
| | | | | | | 2 | semi-upright | Bluecrop, | | | |
| | | | | | | | | Lateblue | | | |
| | | | | | | 3 | spreading | Northland, | | | |
| | | | | | | | | Weymouth | | | |
| 4 | 3 | PQ | One-year-old | Color of middle | Observa- | 1 | green | | | | |
| | | | shoot: color | part of shoot | tion | 2 | greenish red | | | | |
| | | | | extended before | (a) | 3 | greyish red | Briteblue, | | | |
| | | | | dormant period | VG | | | Homebell | | | |
| | | | | | | 4 | reddish yellow | Berkeley, | 5 | 4 | 4 |
| | | | | | | | | Dixi | | | |
| | | | | | | 5 | reddish brown | Blueray, | | | |
| | | | | | | | | Darrow, | | | |
| | | | | | | | | Weymouth | | | |
| | | | | | | 6 | dark red | | | | |
| 5 | | QN | One-year-old | Length of middle | Measure- | 3 | short | | | | |
| | | | shoot: length | part of shoot | ment | 5 | medium | | 5 | 7 | 7 |
| | | | | extended before | mm | 7 | long | | (164 | (222 | (245 |
| | | | | domant period | (a) | | | | mm) | mm) | mm) |
| | | | | | VG | | | | | | |
| 6 | 4 | QN | One-year-old | Length of | Observa- | 3 | short | Avonblue, | | | |
| | | | shoot: length | internode of shoot | tion | | | Weymouth | | | |
| | | | of internode | extended before | (a) | 5 | medium | | 4 | 5 | 5 |
| | | | (upper half) | dormant period | VG | 7 | long | Jersey | (16.2 | (17.0 | (18.0 |
| | | | | (upper half) | | | | | mm) | mm) | mm) |
| 7 | 5 | QN | Leaf: length | Length of leaf | Measure- | 3 | short | | | | |
| | (*) | | | sufficiently | | 5 | medium | | | | |
| | | | | expanded | mmment | 7 | long | | 3 | 5 | 5 |
| | | | | | (b) | | | | (54.0 | (76.0 | (71.0 |
| | | | | | MS/ | | | | mm) | mm) | mm) |
| | | | | | VG | | | | | | |
| 8 | 6 | QN | Leaf: width | Maximum width of | Measure- | 3 | narrow | | | | |
| | | | | mature leaf | ment | 5 | medium | | 5 | 5 | 5 |
| | | | | | mm | 7 | broad | | (33.0 | (37.0 | (36.0 |
| | | | | | (b) | | | | mm) | mm) | |
| | | | | | MS/ | | | | | | mm) |
| | | | | | VG | | | | | | |
| 9 | 7 | QN | Leaf: ratio length/ | Ratio of leaf length to | Measure- | 3 | small | | | | |
| | | | width | maximum width | ment | 5 | medium | | 4 | 5 | 5 |
| | | | | (leaf length/ | (b) | 7 | large | | (1.64) | (2.05) | (1.97) |
| | | | | leaf width) | MS/ | | | | | | |
| | | | | | VG | | | | | | |
| 10 | 8 | PQ | Leaf: shape | Shape of mature leaf | Observa- | 1 | lanceolate | | | | |
| | (*) | | | | tion | 2 | ovate | Northland | | | |
| | | | | | (b) | 3 | elliptic | Berkeley, | 2 | 3 | 3 |
| | | | | | VG | | | Collins, | | | |
| | | | | | | | | Coville | | | |
| | | | | | | 4 | oblong | | | | |
| 11 | | QN | Leaf: shape of tip | Shape of tip of | Observa- | 3 | acute | Weymouth, | | | |
| | | (+) | | mature leaf | tion | | | Woodard | | | |
| | | | | | (b) | 5 | medium | Earliblue, | | | |
| | | | | | VG | | | Tifblue | 5 | 6 | 5 |
| | | | | | | 7 | obtude | Berkeley, | | | |
| | | | | | | | | Climax, | | | |
| | | | | | | | | Southland | | | |
| 12 | 9 | QL | Leaf: color | Color of surface of | Observa- | 1 | yellow | | | | |
| | | | of upper | mature leaf | tion | 2 | green | Bluechip, | 2 | 2 | 2 |
| | | | side | | (b) | | | Bluecrop, | | | |
| | | | | | VG | | | Blueray | | | |
| 13 | 10 | QN | Only varieties | Intensity of green | Observa- | 3 | light | | | | |
| | (*) | | with green leaf | color on surface of | tion | 5 | medium | | | | |
| | | | color: Leaf: | mature leaf | (b) | 7 | dark | | | | |
| | | | intensity of | | VG | | | | 5 | 7 | 7 |
| | | | green color | | | | | | | | |
| | | | on upper side | | | | | | | | |
| 14 | 11 | QL | Leaf: margin | Type of margin of | Observa- | 1 | entire | | | | |
| | (*) | | | mature leaf | tion | 2 | serrate | | 1 | 1 | 1 |
| | | | | | (b) | | | | | | |
| | | | | | VG | | | | | | |
| 15 | 12 | QN | Flower bud: | Intensity of | Observa- | 3 | weak | | | | |
| | | | anthocyanin | antocyanin | tion | 5 | medium | | | | |
| | | | coloration | coloration of flower | (b) | 7 | strong | | 5 | 5 | 5 |
| | | | | bud occurring to one | VG | | | | | | |
| | | | | year old shoot | | | | | | | |
| 16 | 13 | QN | Inflorescence: | Length of | Measure- | 3 | short | | | | |
| | | | length | inflorescence at | ment | 5 | medium | | | | |
| | | | (excluding | flowering time | mm | 7 | long | | 7 | 5 | 5 |
| | | | peduncle) | (excluding peduncle) | (c) | | | | (35.7 | (25.2 | (26.4 |
| | | | | | MS/ | | | | mm) | mm) | mm) |
| | | | | | VG | | | | | | |
| 17 | 14 | PQ | Flower: | Shape of corolla at | Observa- | 1 | urceolate | Bluecrop, | | | |
| | | | shape of | full bloom | tion | | | Jersey | | | |
| | | | corolla | | (c) | 2 | campanulate | Northblue, | 2 | 1-2 | 2 |
| | | | | | VG | | | Northsky | | | |
| | | | | | | 3 | cylindrical | | | | |
| 18 | | PQ | Flower: color of | Color of corolla at full | Observa- | 1 | white | Aliceblue, | | | |
| | | | corolla | bloom | tion | | | Bluetta, | | | |
| | | | | | (c) | | | Briteblue | | | |
| | | | | | VG | 2 | creamy white | Avonblue, | | | |
| | | | | | | | | Berkeley, | | | |
| | | | | | | | | Bluerop | | | |
| | | | | | | 3 | greenish white | Blueray, | 2 | 2-3 | 2-3 |
| | | | | | | | | Collins, | | | |
| | | | | | | | | Coville | | | |
| | | | | | | 4 | light pink | BLuebell, | | | |
| | | | | | | | | Delite, | | | |
| | | | | | | | | Dixi, | | | |
| | | | | | | | | Tifblue | | | |
| 19 | 15 | QN | Flower: size of | Size of corolla | Observa- | 3 | small | | | | |
| | (*) | | corolla tube | tube at full bloom | tion | 5 | medium | | 5 | 5 | 6 |
| | | | | | (c) | 7 | large | | | | |
| | | | | | VG | | | | | | |
| 20 | 16 | QN | Flower: | Intensity of | Observa- | 1 | absent or | | | | |
| | (*) | | anthocyanin | anthocyanin | tion | | very weak | | | | |
| | | | coloration of | coloration on | (c) | 3 | weak | | | | |
| | | | corolla tube | surface of corolla | VG | 5 | medium | | 1 | 1 | 3 |
| | | | | tube | | 7 | strong | | | | |
| 21 | 17 | QL | Flower: | Presence or absence | Observa- | 1 | absent | Herbert | | | |
| | | | ridges on | of ridges on corolla | tion | 9 | present | Aliceblue | | | |
| | | | corolla tube | tube | (c) | | | Berkeley, | 9 | 9 | 9 |
| | | | | | VG | | | Dixi | | | |
| 22 | 18 | QN | Fruit cluster: | Density of fruit per | Observa- | 3 | sparse | Homebell, | | | |
| | | | density | fruit cluster | tion | | | Jersey, | | | |
| | | | | | (d) | | | Woodard | | | |
| | | | | | VG | 5 | medium | Bluechip, | | | |
| | | | | | | | | Bluecrop, | 5 | 5 | 5 |
| | | | | | | | | Bluetta | | | |
| | | | | | | 7 | dense | Darrow, | | | |
| | | | | | | | | Herbert, | | | |
| | | | | | | | | Patriot | | | |
| 23 | 19 | QN | Unripe fruit: | Intensity of green | Observa- | 3 | light | | | | |
| | (*) | | intensity of | color of fruit before | tion | 5 | medium | | 5 | 3 | 5 |
| | | | green color | ripening | VG | 7 | dark | | | | |
| 24 | 20 | QN | Fruit size | Size of fruit at | Observa- | 3 | small | Homebell, | | | |
| | (*) | | | ripening | tion | | | June, | | | |
| | | | | | (d) | | | Northblue | | | |
| | | | | | VG | 5 | medium | Collins, | 7 | 7 | 7 |
| | | | | | | | | Earliblue | (3.79 g) | (4.56 g) | (3.96 g) |
| | | | | | | 7 | large | Berkeley, | | | |
| | | | | | | | | Bluecrop, | | | |
| | | | | | | | | Spartan | | | |
| 25 | 21 | PQ | Fruit: shape in | Shape in | Observa- | 1 | elliptic | | | | |
| | (*) | (+) | longitudinal | longitudinal | tion | 2 | round | Berkeley, | | | |
| | | | section | section of fruit at | (d) | | | Jersey, | | | |
| | | | | ripening | VG | | | Sharpblue | 3 | 3 | 3 |
| | | | | | | 3 | oblate | Earliblue, | | | |
| | | | | | | | | Harison, | | | |
| | | | | | | | | Woodard | | | |
| 26 | | QN | Fruit: size of scar | Size of stem scar | Observa- | 3 | small | | | | |
| | | (+) | | of mature fruit | tion | 5 | medium | | 5 | 5 | 5 |
| | | | | | (d) | 7 | large | | (2.92 | (2.30 | (1.90 |
| | | | | | VG | | | | | | |
| 27 | | PQ | Fruit: shape of | Shape of calyx | Observa- | 1 | star | | mm) | mm) | mm) |
| | | (+) | calyx cavity | cavity of mature | tion | 2 | circular | | 2 | 2 | 2 |
| | | | | fruit | (d) | | | | | | |
| | | | | | VG | | | | | | |
| 28 | 22 | QN | Fruit: attitude of | Attitude of sepals | Observa- | 1 | erect | | | | |
| | | | sepals | relative to mature | tion | 2 | erect to | | | | |
| | | | | fruit | (d) | | semi-erect | | | | |
| | | | | | VG | 3 | semi-erect | | 2 | 1 | 1 |
| | | | | | | 4 | level | | | | |
| 29 | 23 | QN | Fruit: type of | Direction of curving | Observa- | 1 | incurving | | | | |
| | | | sepals | of sepals | tion | 2 | straight | | 3 | 1 | 3 |
| | | | | | (d) | 3 | reflexed | | | | |
| | | | | | VG | | | | | | |
| 30 | 24 | QN | Fruit: diameter | Diameter of calyx | Observa- | 3 | small | Avonblue, | | | |
| | | | of calyx basin | basin of mature fruit | tion | | | Bluechip, | | | |
| | | | | | (d) | | | Sharpblue | | | |
| | | | | | VG | 5 | medium | Blueray, | 5 | 5 | 5 |
| | | | | | | | | Woodard | (5.86 | (5.80 | (5.90 |
| | | | | | | 7 | large | Coville, | mm) | mm) | mm) |
| | | | | | | | | Darrow, | | | |
| | | | | | | | | Homebell | | | |
| 31 | 25 | QN | Fruit: depth of | Depth of calyx basin | Observa- | 3 | shallow | Bluecrop, | | | |
| | | | calyx basin | of mature fruit | tion | | | Rancocas, | | | |
| | | | | | (d) | | | Tifblue | | | |
| | | | | | VG | 5 | medium | Earliblue, | 7 | 7 | 5 |
| | | | | | | | | Jersey | (2.72 | (2.20 | (1.50 |
| | | | | | | 7 | deep | Blueray, | mm) | mm) | mm) |
| | | | | | | | | Collins, | | | |
| | | | | | | | | Dixi | | | |
| 32 | 26 | QN | Fruit: | Intensity of bloom | Observa- | 1 | very weak | | | | |
| | (*) | | intensity | on surface of | tion | 3 | weak | Dixi, | | | |
| | | | of bloom | mature fruit | (d) | | | Herbert, | | | |
| | | | | | VG | | | Sharpblue, | | | |
| | | | | | | 5 | medium | Collins, | 5 | 5 | 5 |
| | | | | | | | | Coville | | | |
| | | | | | | 7 | strong | Avonblue, | | | |
| | | | | | | | | Bluecrop, | | | |
| | | | | | | | | Tifblue | | | |
| 33 | 27 | PQ | Fruit: color of skin | Color of skin of | Observa- | 1 | light blue | Berkeley, | | | |
| | (*) | G | (after removal of | mature fruit after | tion | | | BLuechip, | | | |
| | | | bloom) | removal of bloom | (d) | | | Tifblue | | | |
| | | | | | VG | 2 | medium blue | Blueray, | | | |
| | | | | | | | | Jersey, | 2 | 2 | 2 |
| | | | | | | | | June | | | |
| | | | | | | 3 | dark blue | Dixi, | | | |
| | | | | | | | | Homebell | | | |
| | | | | | | 4 | blue red | | | | |
| 34 | 28 | QN | Fruit: formness | Formness of mature | Observa- | 3 | soft | Herbert, | | | |
| | | (+) | | fruit | tion | | | Homebell, | | | |
| | | | | | (d) | | | Spartan | | | |
| | | | | | VG/ | 5 | medium | Collins, | | | |
| | | | | | VS | | | Dixi, | 5 | 3 | 5 |
| | | | | | | 7 | firm | Coville, | | | |
| | | | | | | | | Southland | | | |
| | | | | | | 9 | very firm | | | | |
| 35 | | PQ | Fruit: color of | Color of flesh of | Observa- | 1 | white | Berkeley, | | | |
| | | | flesh | mature fruit | tion | | | Bluecrop, | | | |
| | | | | | (d) | | | Blueray | | | |
| | | | | | VG | 2 | cream | Earliblue | | | |
| | | | | | | 3 | light green | Bluechip, | 1 | 2-3 | 1-2 |
| | | | | | | | | Lateblue, | | | |
| | | | | | | | | Sharpblue, | | | |
| | | | | | | 4 | light purple | Aliceblue, | | | |
| | | | | | | | | Delite, | | | |
| | | | | | | | | Homebell | | | |
| 36 | 29 | QN | Fruit: sweetness | Sweetness of mature | Observa- | 3 | low | Avonblue, | | | |
| | (*) | (+) | | fruit | tion | | | Bluechip, | | | |
| | | | | | (d) | 5 | medium | Berkeley, | | | |
| | | | | | VG | | | Bluetta, | 7 | 5 | 5 |
| | | | | | | | | Spartan | (Bx | (Bx | (Bx |
| | | | | | | 7 | high | Alicblue, | 14.5) | 11.9) | 11.1) |
| | | | | | | | | Bluecrop, | | | |
| | | | | | | | | Blueray | | | |
| 37 | 30 | QN | Fruit: acidity | Acidity of mature fruit | Observa- | 3 | low | Earliblue, | | | |
| | (*) | (+) | | | tion | | | Homebell | | | |
| | | | | | (d) | 5 | medium | Blueray, | 7 | 5 | 3 |
| | | | | | VG | | | Herbert | (pH | (pH | (pH |
| | | | | | | 7 | high | Collins, | 2.59) | 3.12) | 3.87) |
| | | | | | | | | Elliott, | | | |
| | | | | | | | | Lateblue | | | |
| 38 | 31 | QL | Plant: fruiting type | Shhots to which fruit | Observa- | 1 | on one-year-old | | | | |
| | (*) | G | | adnate | tion | | shoots only | | | | |
| | | | | | (c) | 2 | on one-year- | | 1 | 1 | 1 |
| | | | | | VG | | old and | | | | |
| | | | | | | | current season's | | | | |
| | | | | | | | shoots | | | | |
| 39 | | QN | Fruit: tendency of | Tendency of | Observa- | 3 | less | Earliblue, | | | |
| | | | cracking | cracking during | tion | | | Herbert, | | | |
| | | | | harvest season | (d) | | | Spartan | | | |
| | | | | | VG | 5 | medium | Avonblue, | | | |
| | | | | | | | | Berkeley, | 3 | 3 | 3 |
| | | | | | | | | Bluechip | | | |
| | | | | | | 7 | much | Briteblue, | | | |
| | | | | | | | | Climax, | | | |
| | | | | | | | | Darrow | | | |
| 40 | 32 | QN | Time of | Time to beginning to | Measure- | 3 | early | Avonblue, | | | |
| | (*) | (+) | vegetative bud | burst firstbegetative | ment | | | Beckyblue | | | |
| | | | burst | bud of each | MG | | | Sharpblue | | | |
| | | | | individual plant | | 5 | medium | Darrow, | 5 | 7 | 5 |
| | | | | | | | | Weymouth | April 6 | April 8 | April 5 |
| | | | | | | 7 | late | Elliott, | (2016) | (2016) | (2016) |
| | | | | | | | | Lateblue | | | |
| 41 | 33 | QN | Time of beginning | Time of 10% | Measure- | 1 | very early | | | | |
| | (*) | (+) | of flowering on | flowering occurring to | ment | 3 | early | | | | |
| | | G | one-yearold shoot | one year old shoot | MG | 5 | medium | Bluecrop, | | | |
| | | | | | | | | Collins, | 3 | 3 | 3 |
| | | | | | | | | Woodard | April 25 | April 24 | April 24 |
| | | | | | | 7 | late | Dixi, | (2016) | (2016) | (2016) |
| | | | | | | | | Herbert, | | | |
| | | | | | | | | Lateblue | | | |
| | | | | | | 9 | very late | | | | |
| 42 | 34 | QN | Varieties which fruit | Time of 10% | Measure- | 3 | early | | | | |
| | (*) | (+) | on one-year-old | flowering occurring to | ment | 5 | medium | | | | |
| | | G | shoot and current | current year's shoot | MG | 7 | late | | | | |
| | | | season's shoot: | | | | | | | | |
| | | | Time of beginning | | | | | | — | — | — |
| | | | of flowering on | | | | | | | | |
| | | | current year's | | | | | | | | |
| | | | shoot | | | | | | | | |
| 43 | 35 | QN | Time of beginning | Time of 10% fruit | Measure- | 1 | very early | | | | |
| | | (+) | of fruit ripening on | ripening on one year | ment | 3 | early | Avonblue, | | | |
| | | G | one-year-old shoot | old shoot | MG | | | Earliblue, | | | |
| | | | | | | | | Weymouth | | | |
| | | | | | | 5 | medium | Herbert, | 3 | 3 | 3 |
| | | | | | | | | Jersey | Around | Around | Around |
| | | | | | | 7 | late | Briteblue, | June 20 | June 25 | June 20 |
| | | | | | | | | Elliott, | | | |
| | | | | | | | | Tifblue | | | |
| | | | | | | 9 | very late | | | | |
| 44 | | QN | Period of harvest | Length of harvest | Measure- | 3 | short | Darrow, | | | |
| | | | | period of fruit | ment | | | Northblue | | | |
| | | | | | MG | 5 | medium | Berkeley, | | | |
| | | | | | | | | Bluecrop | 5 | 5 | 5 |
| | | | | | | 7 | loing | Briteblue, | | | |
| | | | | | | | | Tifblue, | | | |
| | | | | | | | | Woodard | | | |
| 45 | 36 | QN | Varieties which fruit | Time of 10% fruit | Measure- | 3 | early | | | | |
| | (*) | (+) | on one-year-old | ripening on current | ment | 5 | medium | | | | |
| | | G | shoot and current | year's shoot | MG | 7 | late | | | | |
| | | | season's shoot: | | | | | | | | |
| | | | Time of beginning | | | | | | — | — | — |
| | | | of ripening on | | | | | | | | |
| | | | current year's | | | | | | | | |
| | | | shoot | | | | | | | | |

What is claimed is:

1. A new and distinct variety of Vaccinium corymbosum L. plant named ‘RYOKU NH-12’, as described and illustrated herein.
